(19) United States
(12) Patent Application Publication
(10) Pub. No.: US 2010/0273868 A1
(43) Pub. Date: Oct. 28, 2010

(54) R-Zileuton for Use in Conditions Associated with Increased 5-Lipoxygenase and/or Leukotriene Activity (EG Asthma)

(75) Inventors: Walter Newman, Boston, MA (US); Anjali Kumar, Belmont, MA (US); Lynne Libertine, Framingham, MA (US)

Correspondence Address: FOLEY & LARDNER LLP, 111 HUNTINGTON AVENUE, 26TH FLOOR, BOSTON, MA, 02199-7610, US

(73) Assignee: Cornerstone Therapeutics Inc.

(21) Appl. No.: 12/521,965

(22) PCT Filed: Jan. 4, 2008
(86) PCT No.: PCT/US2008/000101

§ 371 Date: Jun. 2, 2010

Related U.S. Application Data
(60) Provisional application No. 60/879,273, filed on Jan. 5, 2007.

(51) Int. Cl. A61K 31/381; A61P 11/06; A61P 19/02; A61P 17/06; A61P 19/06; A61P 17/10; A61P 35/00; A61P 9/10; A61P 1/00

(52) U.S. Cl. 514/443

(57) ABSTRACT

The invention pertains to a method of treating a condition mediated by or characterized by increased 5-lipoxygenase activity like for instance asthma in a patient suffering therefrom comprising administering to said patient a composition comprising (R)-zileuton and a pharmaceutically acceptable excipient, wherein said composition is substantially free of (S)-zileuton.

### RELATED APPLICATIONS

This application claims the benefit of U.S. Provisional Application No. 60/879,273, filed on Jan. 5, 2007, the entire teachings of which are incorporated herein by reference.

### BACKGROUND OF THE INVENTION

The enzyme 5-lipoxygenase catalyzes the first step in the metabolism of arachidonic acid in a pathway that produces 5-hydroxyeicosatetraenoic acid (5-HETE) as well as the leukotrienes. Leukotrienes (including, for example, LTB₄, LTC₄, LTD₄ and LTE₄) have been recognized to have several biological effects including the induction of neutrophil and eosinophil migration, leukocyte adhesion, increased capillary permeability, and smooth muscle contraction which in turn contribute to inflammation, edema, mucus secretion, and bronchoconstriction. Accordingly, compounds that inhibit 5-lipoxygenase activity are useful in the treatment and/or prevention of diseases or conditions associated with such biological effects.

One compound that inhibits 5-lipoxygenase is zileuton ((±)-1-(1-Benzo[b]thien-2-ylethyl)-1-hydroxyurea). Zileuton has the following chemical structure:

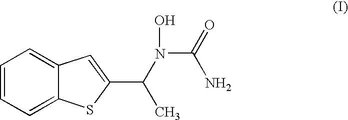

A tablet comprising 600 milligrams (mg) of zileuton (ZYFLO® and Zyflo® CR zileuton tablets) is currently marketed for the treatment of asthma. Zileuton has also been reported as effective in treating patients suffering from sickle cell anemia, nasal polyposis, acne, systemic lupus erythematosus, atopic dermatitis, allergy and rheumatoid arthritis, among others (Zouboulis et al., Arch. Dermatol., 139(5):668-70 (2003); Parnes et al., Ear Nose Throat J. 79(1):18-20, 24-5 (2000); U.S. Pat. No. 7,026,344; Hakshaw et al., J. Rheumatol. 22(3):462-8 (1998); Woodmansee et al., 83(6 Pt 1):548-52 (1999); Kane et al., 97(2):646-54, (1996); Weinblatt et al., 19(10):1537-41 (1992); Willemsen et al., 31(1):1-3 (2000)). Additionally, in animal studies, zileuton has shown effectiveness in inhibiting tumor growth and in the treatment of multiple organ injury caused by endotoxemia, renal ischemia-reperfusion injury, experimental colitis, lipopolysaccharide-induced hypothermia, respiratory syncytial virus and acute vesicular stomatitis virus (VSV) encephalitis (Collin et al, J Leukoc Biol. 2004 November; 76(5):961-70, Patel et al., 66(2):220-7 (2004), Mazzon et al., Shock, 25(4):377-83 (2006), Singh et al., Indian J Exp Biol., 43(12):1150-5 (2005), Welliver et al., J Infect Dis. 187(11):1773-9 (2003), Chen et al., 120(1-2):94-102 (2001), Hussey et al., Br J. Cancer. 74(5):683-7 (1996)). Furthermore, the ability of zileuton to prevent lung cancer is currently under clinical trial investigation (http://www.clinicaltrials.gov/ct/show/NCT00056004).

In its currently marketed form, zileuton is administered as a racemic mixture of R(+) and S(−) enantiomers. The current recommended dosing regimen for zileuton is administration four times a day for a total daily dose of 2400 mg (as well as twice daily Zyflo® CR at 1200 mg each dose). It would be advantageous to develop methods of inhibiting 5-lipoxygenase activity by administering zileuton at a lower dose and/or at a less frequent dosing interval than racemic zileuton is currently administered.

### SUMMARY OF THE INVENTION

It has now surprisingly been found that (R)-zileuton is more efficacious than either of (S)-zileuton and racemic zileuton in inhibiting 5-lipoxygenase activity. For example, it has been shown that (R)-zileuton exhibits twelve-fold greater potency in inhibiting the production of LTB₄ in human whole blood than (S)-zileuton (see Example 3 below). Moreover, in a BALB/c murine model of allergic lung inflammation, (R)-zileuton administered at once a day oral dose of either 2 or 4 mg/kg exhibited significantly greater efficacy than racemic zileuton (Example 5). A four times daily at 10 mg/kg dosing is typically used in this BALB/c murine model of allergic lung inflammation to achieve maximal efficacy and to approximate human therapeutic dosage.

Accordingly, the present invention pertains to a method of treating a condition mediated by or characterized by increased 5-lipoxygenase activity in a patient suffering therefrom comprising administering to said patient a composition comprising (R)-zileuton and a pharmaceutically acceptable excipient, wherein said composition is substantially free of (S)-zileuton. In one embodiment, said (R)-zileuton is administered at a total daily dose from about 450 mg to about 1200 mg per day. In another embodiment, said (R)-zileuton is administered at a total daily dose from about 500 mg to about 1000 mg per day. In yet another embodiment, said (R)-zileuton is administered at a total daily dose from about 600 mg to about 900 mg per day. In another embodiment, said (R)-zileuton is administered at a total daily dose of about 600 mg per day. In yet another embodiment, said (R)-zileuton is administered at a total daily dose of about 900 mg per day. In an additional embodiment, said (R)-zileuton is administered at a total daily dose of about 1000 mg per day. In another embodiment, said (R)-zileuton is administered at a total daily dose of about 1200 mg per day. In a further embodiment, said total daily dose is administered as a single dose. In yet another embodiment, the total daily dose is administered as two doses.

In an additional embodiment, the invention is a method of treating a condition mediated by or characterized by increased leukotriene activity in a patient suffering therefrom comprising administering to said patient a composition comprising (R)-zileuton and a pharmaceutically acceptable excipient, wherein said composition is substantially free of (S)-zileuton. In one embodiment, said (R)-zileuton is administered at a total daily dose from about 450 mg to about 1200 mg per day. In another embodiment, said (R)-zileuton is administered at a total daily dose from about 500 mg to about 1000 mg per day. In yet another embodiment, said (R)-zileuton is administered at a total daily dose from about 600 mg to about 900 mg per day. In another embodiment, said (R)-zileuton is administered at a total daily dose of about 600 mg per day. In yet another embodiment, said (R)-zileuton is administered at a total daily dose of about 900 mg per day. In an additional embodiment, said (R)-zileuton is administered at a total daily dose of about 1000 mg per day. In another embodiment, said (R)-zileuton is administered at a total daily dose of about 1200 mg per day. In a further embodiment, said total daily dose is administered as a single dose. In yet another embodiment, the total daily dose is administered as two doses.

In a further embodiment, the invention is directed to a method of treating an inflammatory condition in a patient suffering therefrom comprising administering to said patient a composition comprising (R)-zileuton and a pharmaceutically acceptable excipient, wherein said composition is substantially free of (S)-zileuton. In one embodiment, said (R)-zileuton is administered at a total daily dose from about 450 mg to about 1200 mg per day. In another embodiment, said (R)-zileuton is administered at a total daily dose from about 500 mg to about 1000 mg per day. In yet another embodiment, said (R)-zileuton is administered at a total daily dose from about 600 mg to about 900 mg per day. In another embodiment, said (R)-zileuton is administered at a total daily dose of about 600 mg per day. In yet another embodiment, said (R)-zileuton is administered at a total daily dose of about 900 mg per day. In an additional embodiment, said (R)-zileuton is administered at a total daily dose of about 1000 mg per day. In another embodiment, said (R)-zileuton is administered at a total daily dose of about 1200 mg per day. In a further embodiment, said total daily dose is administered as a single dose. In yet another embodiment, the total daily dose is administered as two doses.

In yet another embodiment, the invention is directed to a method of treating a condition in a patient suffering therefrom comprising administering to said patient a composition comprising (R)-zileuton and a pharmaceutically acceptable excipient, wherein said composition is substantially free of (S)-zileuton and wherein said condition is selected from the group consisting of asthma, rheumatoid arthritis, gout, psoriasis, allergy, rhinitis, adult respiratory distress syndrome, chronic obstructive pulmonary disease, acne, atopic dermatitis, conjunctivitis, ischemia/reperfusion injury, atherosclerosis, aortic aneurysm, nasal polyposis, inflammatory bowel disease, irritable bowel syndrome, cancer, tumor, respiratory syncytial virus, Sjogren-Larsson syndrome, sickle cell disease, sepsis, endotoxin shock, myocardial infarction and stroke.

In another embodiment, the invention is directed to a method of treating a condition in a patient suffering therefrom comprising administering to said patient a composition comprising (R)-zileuton and a pharmaceutically acceptable excipient, wherein said composition is substantially free of (S)-zileuton and wherein said condition is selected from the group consisting of asthma, rheumatoid arthritis, gout, psoriasis, allergy, rhinitis, adult respiratory distress syndrome, chronic obstructive pulmonary disease, acne, atopic dermatitis, ischemia/reperfusion injury, atherosclerosis, aortic aneurysm, nasal polyposis, inflammatory bowel disease, irritable bowel syndrome, cancer, tumor, respiratory syncytial virus, Sjogren-Larsson syndrome, sickle cell disease, sepsis, endotoxin shock, myocardial infarction and stroke.

In one embodiment, said (R)-zileuton is administered at a total daily dose from about 450 mg to about 1200 mg per day. In another embodiment, said (R)-zileuton is administered at a total daily dose from about 500 mg to about 1000 mg per day. In yet another embodiment, said (R)-zileuton is administered at a total daily dose from about 600 mg to about 900 mg per day. In another embodiment, said (R)-zileuton is administered at a total daily dose of about 600 mg per day. In yet another embodiment, said (R)-zileuton is administered at a total daily dose of about 900 mg per day. In an additional embodiment, said (R)-zileuton is administered at a total daily dose of about 1000 mg per day. In another embodiment, said (R)-zileuton is administered at a total daily dose of about 1200 mg per day. In a further embodiment, said total daily dose is administered as a single dose. In yet another embodiment, the total daily dose is administered as two doses.

In yet another embodiment, the invention is a method of treating moderate persistent asthma or severe persistent asthma comprising administering to said patient a composition comprising (R)-zileuton and a pharmaceutically acceptable excipient, wherein said composition is substantially free of (S)-zileuton. In one embodiment, said (R)-zileuton is administered at a total daily dose from about 450 mg to about 1200 mg per day. In another embodiment, said (R)-zileuton is administered at a total daily dose from about 500 mg to about 1000 mg per day. In yet another embodiment, said (R)-zileuton is administered at a total daily dose from about 600 mg to about 900 mg per day. In another embodiment, said (R)-zileuton is administered at a total daily dose of about 600 mg per day. In yet another embodiment, said (R)-zileuton is administered at a total daily dose of about 900 mg per day. In an additional embodiment, said (R)-zileuton is administered at a total daily dose of about 1000 mg per day. In another embodiment, said (R)-zileuton is administered at a total daily dose of about 1200 mg per day. In a further embodiment, said total daily dose is administered as a single dose. In yet another embodiment, the total daily dose is administered as two doses.

### BRIEF DESCRIPTION OF THE DRAWINGS

FIG. 1 is a graph showing the mean plasma concentration (ng/ml) of zileuton over time (minutes) following an oral dose of 10 milligrams per kilogram (mpk) racemic zileuton or 5 mpk (R)-zileuton over time (minutes) to dogs. Also shown are the concentrations of the metabolite (A66193) of racemic zileuton and (R)-zileuton.

FIG. 2A is a graph showing the mean plasma concentration (ng/ml) of zileuton and its metabolite and percent inhibition of LTB₄ production over time (minutes) following an oral dose of 10 mpk of racemic zileuton to dogs.

FIG. 2B is a graph showing the mean plasma concentration (ng/ml) or zileuton and its metabolite and percent inhibition of LTB₄ production over time (minutes) following an oral dose of 5 mpk (R)-zileuton to dogs.

FIG. 3 is a graph showing percent inhibition of LTB₄ production in human whole blood in vitro versus concentration (μM) of racemic zileuton, (R)-zileuton and (S)-zileuton.

FIG. 4 is a plot showing total cell count per mL of bronchoalveolar lavage fluid in mice challenged by ovalbumin and treated by R-, S- or racemic zileuton (see Example 5).

FIG. 5A and FIG. 5B are plots showing eosinophil (A) or neutrophil (B) counts per mL of bronchoalveolar lavage fluid in mice challenged by ovalbumin and treated by R—, S— or racemic zileuton (see Example 5).

### DETAILED DESCRIPTION OF THE INVENTION

As used herein, “a” or “an” are taken to mean one or more unless otherwise specified.

Zileuton has the chemical structure described above in Formula (I) with one asymmetric center. Zileuton exists as a pair of enantiomers referred to herein as (R)-zileuton and (S)-zileuton. The structure of (R)-zileuton or (+)-zileuton is shown below in Formula (II):

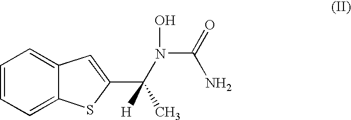

The structure of (S)-zileuton or (−)-zileuton is shown below in Formula (III):

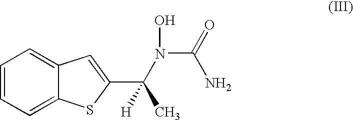

The methods of the present invention comprise administering to a patient a composition comprising zileuton substantially free of (S)-zileuton. As used herein, zileuton substantially free of (S)-zileuton is (R)-zileuton. The phrases “zileuton substantially free of (S)-zileuton” and “(R)-zileuton substantially free of (S)-zileuton” are used interchangeably herein. A composition comprising zileuton is substantially free of (S)-zileuton if at least 80% by weight of the zileuton in the composition is (R)-zileuton, and 20% or less by weight of the zileuton in the composition is (S)-zileuton. In another embodiment, the composition is substantially free of (S)-zileuton if at least 85% by weight of the zileuton is (R)-zileuton and 15% or less by weight of the zileuton is (S)-zileuton. In yet another embodiment, the composition is substantially free of (S)-zileuton if at least 90% by weight of the zileuton is (R)-zileuton and 10% or less by weight of the zileuton is (S)-zileuton. In another embodiment, the composition is substantially free of (S)-zileuton if at least 95% by weight of the zileuton is (R)-zileuton and 5% or less by weight of the zileuton is (S)-zileuton. In yet another embodiment, the composition is substantially free of (S)-zileuton if at least 97% by weight of the zileuton is (R)-zileuton and 3% or less by weight of the zileuton is (S)-zileuton. In a further embodiment, the composition is substantially free of (S)-zileuton if at least 99% by weight of the zileuton is (R)-zileuton and 1% or less by weight of the zileuton is (S)-zileuton. In another embodiment, the method of the present invention comprises administering a composition comprising optically pure (R)-zileuton or (R)-zileuton that is substantially optically pure. The (R)-zileuton is “substantially optically pure” if at least 95% by weight of the zileuton in composition is (R)-zileuton. In another embodiment, (R)-zileuton is substantially optically pure if it at least 97% by weight of zileuton in the composition is (R)-zileuton. In a further embodiment, the (R)-zileuton is substantially optically pure if it at least 99% by weight of zileuton in the composition is (R)-zileuton. As used herein, “racemic” means a mixture of the (−) and (+) enantiomers or (S)- and (R)-enantiomers of a compound wherein the (−) and (+) enantiomers or (S)— and (R)-enantiomers are present in approximately a 1:1 ratio.

(R)-zileuton may be prepared using chiral synthons or chiral reagents, or resolved using conventional techniques. Methods for the preparation of racemic zileuton have been described, for example, in U.S. Pat. Nos. 4,873,259 and 6,080,874 and by Hisao et al., Tetrahedron Letters, 33(19): 2629-32 (1992). (R)-zileuton can be prepared by the resolution of racemic zileuton, such as by using (4S)-4-benzyl-2-oxazolidinone-3-carbonyl chloride (Garigipati et al., Tetrahedron Letters, 34(35): 5537-40 (1993)). (R)-zileuton can also be chemically resolved using the following: esterification with oxalyl chloride and R-mandelic acid, isolation of the diastereomeric mixture from cold ethyl acetate, hydrolysis of the diastereomer to yield the (R)-zileuton which can then purified by recrystallization. Methods for the enantioselective synthesis of (R)-zileuton have also been described. For example, a method for the preparation of (R)-zileuton using the addition of Grignard reagents to N-glycosyl nitrones has been described (Basha et al., J. Org. Chem., 59(20), 6103-6 (1994)). The enantioselective synthesis of (R)-zileuton has also been described using either L-(+)-lactic acid or a gulofuranose auxiliary (Hsiao et al., 33: 2629-32 (1992); Roloff et al., 35(7): 1011-14 (1994)). Furthermore, a method for the preparation of (R)-zileuton is also described in U.S. Pat. No. 5,663,368.

5-lipoxygenase is the enzyme that catalyzes the conversion of arachidonic acid to 5-hydroperoxyeicosatetraenoic acid (5-HPETE) in a pathway that produces leukotrienes. Leukotrienes are biological molecules produced by the metabolism of arachidonic acid. Leukotrienes are divided into two groups: leukotrienes that play a role in conditions where inflammation is dependent on neutrophils and the cysteinyl leukotrienes (LTC', LTD₄ and LTE₄) which act at their cell surface receptors, cysLT1 and cysLT2, on mast cells and eosinophils and have a role in bronchoconstriction and in the secretion of mucus in respiratory conditions such as asthma. The method of the prevent invention may be used to treat any condition now known or later discovered to be associated with or mediated by increased 5-lipoxygenase activity and/or increased leukotriene activity. Conditions associated with or mediated by increased 5-lipoxygenase activity and/or the increased synthesis of leukotrienes include, but are not limited to, inflammatory conditions as well as other conditions such as sickle cell disease, nasal polyposis, sinusitis, aortic aneurysm, respiratory syncytial virus, VSV encephalitis, tumors and cancer.

In one embodiment, the invention is directed to a method of treating a condition selected from the group consisting of asthma, rheumatoid arthritis, gout, psoriasis, allergy, rhinitis, adult respiratory distress syndrome, chronic obstructive pulmonary disease, acne, atopic dermatitis, conjunctivitis, atherosclerosis, aortic aneurysm, sickle cell disease, acute lung injury, ischemia/reperfusion injury, nasal polyposis, inflammatory bowel disease (including, for example, ulcerative colitis and Crohn's disease), irritable bowel syndrome, cancer, tumors, respiratory syncytial virus, sepsis, endotoxic shock, myocardial infarction, Sjorgen-Larrson syndrome and stroke.

In one embodiment, the invention is directed to a method of treating a condition selected from the group consisting of asthma, rheumatoid arthritis, gout, psoriasis, allergy, rhinitis, adult respiratory distress syndrome, chronic obstructive pulmonary disease, acne, atopic dermatitis, atherosclerosis, aortic aneurysm, sickle cell disease, acute lung injury, ischemia/reperfusion injury, nasal polyposis, inflammatory bowel disease (including, for example, ulcerative colitis and Crohn's disease), irritable bowel syndrome, cancer, tumors, respiratory syncytial virus, sepsis, endotoxic shock, myocardial infarction, Sjorgen-Larrson syndrome and stroke.

In another embodiment, the invention is directed to a method of treating a condition selected from the group consisting of asthma, rheumatoid arthritis, allergic rhinitis, adult respiratory distress syndrome, chronic obstructive pulmonary disease, acne, conjunctivitis, atherosclerosis, inflammatory bowel disease, sickle cell disease, nasal polyposis, sinusitis, aortic aneurysm and stroke.

In yet another embodiment, the invention is directed to a method of treating a condition selected from the group consisting of asthma, rheumatoid arthritis, allergic rhinitis, adult respiratory distress syndrome, chronic obstructive pulmonary disease, acne, atherosclerosis, inflammatory bowel disease, sickle cell disease, nasal polyposis, sinusitis, aortic aneurysm and stroke.

In a further embodiment, the invention is directed to a method of treating a condition selected from the group consisting of asthma, adult respiratory distress syndrome and chronic obstructive pulmonary disease. In yet another embodiment, the invention is directed to a method of treating a condition selected from the group consisting of sickle cell disease, nasal polyposis, atherosclerosis, sinusitis, aortic aneurysm and stroke.

In another embodiment, the invention is directed to a method of treating a condition selected from the group consisting of asthma, rheumatoid arthritis, allergic rhinitis, adult respiratory distress syndrome, chronic obstructive pulmonary disease, acne, atherosclerosis, inflammatory bowel disease, sickle cell disease, nasal polyposis, sinusitis, aortic aneurysm and stroke. In a further embodiment, the invention is directed to a method of treating a condition selected from the group consisting of asthma, adult respiratory distress syndrome and chronic obstructive pulmonary disease. In yet another embodiment, the invention is directed to a method of treating a condition selected from the group consisting of sickle cell disease, nasal polyposis, atherosclerosis, sinusitis, aortic aneurysm and stroke.

In one embodiment, the condition mediated by lipoxygenase and/or leuktoriene activity is an inflammatory condition. Inflammatory conditions include, but are not limited to, appendicitis, peptic, gastric or duodenal ulcers, peritonitis, pancreatitis, acute or ischemic colitis, diverticulitis, epiglottitis, achalasia, cholangitis, cholecystitis, hepatitis, inflammatory bowel disease (including, for example, Crohn's disease and ulcerative colitis), enteritis, Whipple's disease, asthma, chronic obstructive pulmonary disease, acute lung injury, ileus (including, for example, post-operative ileus), allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopic silicovolcanoconiosis, alvealitis, bronchiolitis, pharyngitis, pleurisy, sinusitis, influenza, respiratory syncytial virus, herpes, disseminated bacteremia, Dengue fever, candidiasis, malaria, filariasis, amebiasis, hydatid cysts, burns, dermatitis, dermatomyositis, conjunctivitis, sunburn, urticaria, warts, wheals, acne, vasulitis, angiitis, endocarditis, arteritis, atherosclerosis, thrombophlebitis, pericarditis, myocarditis, myocardial ischemia, periarteritis nodosa, rheumatic fever, Alzheimer's disease, celiac disease, congestive heart failure, adult respiratory distress syndrome, meningitis, encephalitis, multiple sclerosis, cerebral infarction, cerebral embolism, Guillame-Barre syndrome, neuritis, neuralgia, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcet's syndrome, allograft rejection, graft-versus-host disease, Type I diabetes, ankylosing spondylitis, Berger's disease, Type II diabetes, Retier's syndrome, or Hodgkins disease.

In another embodiment, inflammatory conditions include, but are not limited to, appendicitis, peptic, gastric or duodenal ulcers, peritonitis, pancreatitis, acute or ischemic colitis, diverticulitis, epiglottitis, achalasia, cholangitis, cholecystitis, hepatitis, inflammatory bowel disease (including, for example, Crohn's disease and ulcerative colitis), enteritis, Whipple's disease, asthma, chronic obstructive pulmonary disease, acute lung injury, ileus (including, for example, post-operative ileus), allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopic silicovolcanoconiosis, alvealitis, bronchiolitis, pharyngitis, pleurisy, sinusitis, influenza, respiratory syncytial virus, herpes, disseminated bacteremia, Dengue fever, candidiasis, malaria, filariasis, amebiasis, hydatid cysts, burns, dermatitis, dermatomyositis, sunburn, urticaria, warts, wheals, acne, vasulitis, angiitis, endocarditis, arteritis, atherosclerosis, thrombophlebitis, pericarditis, myocarditis, myocardial ischemia, periarteritis nodosa, rheumatic fever, Alzheimer's disease, celiac disease, congestive heart failure, adult respiratory distress syndrome, meningitis, encephalitis, multiple sclerosis, cerebral infarction, cerebral embolism, Guillame-Barre syndrome, neuritis, neuralgia, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcet's syndrome, allograft rejection, graft-versus-host disease, Type I diabetes, ankylosing spondylitis, Berger's disease, Type II diabetes, Retier's syndrome, or Hodgkins disease.

In a further embodiment, the inflammatory condition is selected from the group consisting of asthma, rheumatoid arthritis, chronic obstructive pulmonary disease, inflammatory bowel disease, allergy, organ ischemia, reperfusion injury, rhinitis, dermatitis, atherosclerosis, myocardial ischemia, adult respiratory distress syndrome and cystic fibrosis. In yet another embodiment, the inflammatory condition is selected from the group consisting of asthma, rheumatoid arthritis, chronic obstructive pulmonary disease, allergy, atherosclerosis, adult respiratory distress syndrome and cystic fibrosis. In an additional embodiment, the inflammatory condition is selected from the group consisting of asthma, rheumatoid arthritis, chronic obstructive pulmonary disease, allergy, atherosclerosis, adult respiratory distress syndrome and cystic fibrosis. In a further embodiment, the inflammatory condition is selected from the group consisting of asthma, chronic obstructive pulmonary disease and adult respiratory distress syndrome.

In one embodiment, the methods of the invention can be used to treat asthma. Asthma is classified in four categories: mild intermittent, mild persistent, moderate persistent and severe persistent ((The 2007 Third Expert Panel Report and Guidelines for the Diagnosis and Management of Asthma, sponsored by the National Heart, Lung, and Blood Institute, part of the National Institutes of Health, is available at http://www.nhlbi.nih.gov/guidelines/asthma/asthgdln.htm). A patient suffering from mild intermittent asthma exhibits daytime asthma symptoms two or less times per week and nighttime asthma symptoms two or less times per month. The mild intermittent asthma sufferer also shows a forced expiratory volume in one second (the volume exhaled during the first second of a forced expiratory maneuver started from the level of total lung capacity; FEV₁) of at least 80% of a predicted FEV₁ and/or an expiratory peak flow (the maximum flow generated during expiration performed with maximal force and started after a full inspiration; PEF) of at least 80% of the patient's personal best (the patient's highest peak flow). A patient with mild persistent asthma suffers from daytime asthma symptoms more than twice a week but less than daily and suffers nighttime asthma symptoms two or more times per month. The mild persistent asthma patient has an FEV₁ of 80% or greater and/or a PEF of at least 80% of personal best. A patient suffering from moderate persistent asthma suffers at least daily asthma symptoms and more than one nighttime asthma symptom per week. The moderate persistent asthma patient has an FEV₁ of between 60 and 80% of predicted and/or a PEF of at least 60-80% of personal best. A patient suffering from severe persistent asthma suffers continual daytime asthma symptoms, frequent nighttime symptoms and has limited physical activity. The severe persistent asthma patient has an FEV₁ of 60% or less than predicted and/or a PEF of 60% or less of personal best. In one embodiment, the invention is directed to a method of treating a patient suffering from moderate persistent asthma or severe persistent asthma comprising administering a composition comprising (R)-zileuton substantially free of (S)-zileuton and a pharmaceutically acceptable excipient. In another embodiment, the invention is a method of treating a patient suffering from moderate persistent asthma or severe persistent asthma comprising administering a composition comprising (R)-zileuton substantially free of (S)-zileuton and a pharmaceutically acceptable excipient wherein said (R)-zileuton is administered at a total daily dose from about 250 mg to about 1000 mg per day.

In one embodiment, the present invention is directed to a method for treating a patient suffering from a condition mediated by increased 5-lipoxygenase activity and/or the increased synthesis of leukotrienes comprising administering a composition comprising (R)-zileuton substantially free of (S)-zileuton and a pharmaceutically acceptable excipient wherein said (R)-zileuton is administered at a total daily dose from about 450 mg to about 1200 mg per day. In another embodiment, said (R)-zileuton is administered at a total daily dose from about 500 mg to about 1000 mg per day. In yet another embodiment, said (R)-zileuton is administered at a total daily dose from about 600 mg to about 900 mg per day. In another embodiment, said (R)-zileuton is administered at a total daily dose of about 600 mg per day. In yet another embodiment, said (R)-zileuton is administered at a total daily dose of about 900 mg per day. In an additional embodiment, said (R)-zileuton is administered at a total daily dose of about 1000 mg per day. In another embodiment, said (R)-zileuton is administered at a total daily dose of about 1200 mg per day. In a further embodiment, said total daily dose is administered as a single dose. In yet another embodiment, the total daily dose is administered as two doses. In one embodiment, the treatment comprises starting and maintaining the patient at a disclosed total daily dose. The disclosed daily dose is maintained, for example, for a period of up to 3, 4, 5 or 6 days, or 1, 2, 3 or 4 weeks, or 1, 2, 3, 4, 5, 6, 7, 8, 9, 10 or 11 months or 1, 2 or 3 years. For example, a patient's treatment could be initiated and maintained at a total daily dose from about 450 mg to about 1200 mg per day. In another embodiment, the patient is an adult. In yet another embodiment, the patient is an adult under 65 years of age. In yet another embodiment, the patient has no impairment of renal or hepatic function.

As used herein, “treatment” and/or “treating” refer to therapeutic treatment as well as prophylactic treatment or preventative measures. The excipient included with the compounds of the pharmaceutical compositions of the invention is chosen based on the expected route of administration of the composition in therapeutic applications. The route of administration of the composition depends on the condition to be treated. For example, intravenous injection may be preferred for treatment of a systemic disorder such as endotoxic shock, and oral administration may be preferred to treat a gastrointestinal disorder such as a gastric ulcer. The route of administration and the dosage of the composition to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms.

The composition comprising (R)-zileuton substantially free of (S)-zileuton can be administered by a variety of routes including, but not limited to, parenteral, oral, pulmonary, ophthalmic, nasal, rectal, vaginal, aural, topical, buccal, transdermal, intravenous, intramuscular, subcutaneous, ocular, intradermal, intraocular, intracerebral, intralymphatic, intraarticular, intrathecal and intraperitoneal. In one embodiment the composition is administered orally. The daily dose can be administered as a single dose or as multiple, divided doses. In one embodiment, the daily dose is administered as a single daily dose. In another embodiment, the daily dose is administered as two doses.

In one embodiment, the pharmaceutical composition of the present invention is administered orally. For the purpose of oral therapeutic administration, the pharmaceutical compositions of the present invention may be incorporated with excipients and used in the form of tablets, troches, capsules, elixirs, suspensions, syrups, wafers, chewing gums and the like. Tablets, pills, capsules, troches and the like may also contain binders, excipients, disintegrating agent, lubricants, glidants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and non-toxic in the amounts used. In another embodiment, the composition is administered as a tablet or a capsule.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor, and the like. For vaginal administration, a pharmaceutical composition may be presented as pessaries, tampons, creams, gels, pastes, foams or spray.

In addition to the common dosage forms set out above, the composition of the present invention may also be administered by controlled release means, delivery devices, or both, as are well known to those of ordinary skill in the art, such as those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566. These pharmaceutical compositions can be used to provide slow or controlled-release of the active ingredient therein using, for example, hydropropylmethyl cellulose in varying proportions to provide the desired release profile, other polymer matrices, gels, permeable membranes, osmotic systems, multilayer coatings, microparticles, liposomes, microspheres, or the like, or a combination thereof. The controlled-release of the active ingredient may be stimulated by various inducers, for example pH, temperature, enzymes, water, or other physiological conditions or compounds. The term “controlled-release” in the context of the present invention is defined herein as the inclusion in the pharmaceutical composition of a compound or compounds, including polymers, polymer matrices, gels, permeable membranes, liposomes, microspheres, or the like, or a combination thereof, that facilitates the controlled-release of (R)-zileuton in the pharmaceutical composition.

The present invention includes nasally administering to the mammal a therapeutically effective amount of the composition. As used herein, nasally administering or nasal administration includes administering the composition to the mucus membranes of the nasal passage or nasal cavity of the patient. As used herein, pharmaceutical compositions for nasal administration of a composition include therapeutically effective amounts of the compound prepared by well-known methods to be administered, for example, as a nasal spray, nasal drop, suspension, gel, ointment, cream or powder. Administration of the composition may also take place using a nasal tampon or nasal sponge.

For topical administration, suitable formulations may include biocompatible oil, wax, gel, powder, polymer, or other liquid or solid carriers. Such formulations may be administered by applying directly to affected tissues, for example, a liquid formulation to treat infection of conjunctival tissue can be administered dropwise to the subject's eye, or a cream formulation can be administered to the skin.

The composition of the present invention can be administered parenterally such as, for example, by intravenous, intramuscular, intrathecal or subcutaneous injection. Parenteral administration can be accomplished by incorporating a composition of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the pharmaceutical composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves and the like.

In addition to the usual meaning of administering the formulations described herein to any part, tissue or organ whose primary function is gas exchange with the external environment, for purposes of the present invention, “pulmonary” will also mean to include a tissue or cavity that is contingent to the respiratory tract, in particular, the sinuses. For pulmonary administration, an aerosol formulation containing the active agent, a manual pump spray, nebulizer or pressurized metered-dose inhaler as well as dry powder formulations are contemplated. Suitable formulations of this type can also include other agents, such as antistatic agents, to maintain the disclosed compounds as effective aerosols.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the compound intended for pulmonary administration is dissolved, suspended or emulsified in a mixture of a solvent, surfactant and propellant. The mixture is maintained under pressure in a canister that has been sealed with a metering valve.

### EXEMPLIFICATION

The invention is illustrated by the following examples which are not intended to be limiting in any way.

### Example 1

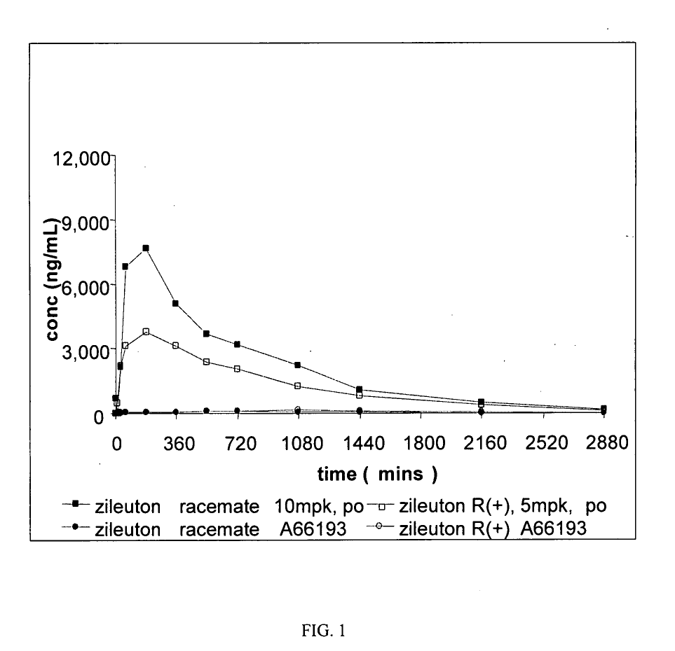

### Mean Plasma Concentration (ng/ml) of Zileuton Over Time After Administration of Racemic or (R)-Zileuton Over Time after Administration to Dogs (Methods for FIG. 1)

The plasma concentration-time profiles for racemic zileuton and (R)-zileuton were evaluated in beagle dogs dosed orally with either 10 mg/kg racemic zileuton or 5 mg/kg (R)-zileuton in gelcaps. Following oral dosing blood samples were collected from the test animals into sodium-EDTA anticoagulant tubes at selected time points up to 48 hours post-dose, processed into plasma and analysed by an LC-MS/MS method for zileuton concentration. The LC-MS/MS method utilized a 0.1% trifluoroacetic in water and methanol gradient HPLC system (initial 45:55 to 10:90% over 4 minutes with a Zorbax SB-C18 4.6×30 mm column at a flow rate of 1.5 mL/min). The retention time of zileuton was 2.0 minutes. A stable labeled version of zileuton was used to internally standardize the analysis. Mass spectrometer analysis by electrospray ionization of the HPLC eluent was performed in SRM mode (ESI 3.5 kV; tube lens voltage 71.0; capillary offset 40.3; L11-24.3; capillary temperature 350° C.; dwell 100 msec; collision energy 15 eV). Plasma concentrations of zileuton in the test samples were estimated by interpolation of sample peak area/height data from a standard concentration-peak area/height plot. The area under the plasma concentration-time (AUC) was calculated by non-compartmental pharmacokinetic analysis (WinNonlin, Pharsight, Mountain View, Calif.) using the trapezoidal rule.

FIG. 1 shows the mean plasma concentration (ng/ml) of racemic zileuton and (R)-zileuton over time (minutes). As shown in FIG. 1, (R)-zileuton comprises 58% of the racemic zileuton AUC (area under the curve). The AUC of racemic zileuton at 10 mg/kg administered orally (po) is 87 μg/ml*hr versus and AUC of 52 μg/ml*hr for (R)-zileuton at 5 mg/kg, po.

### Example 2

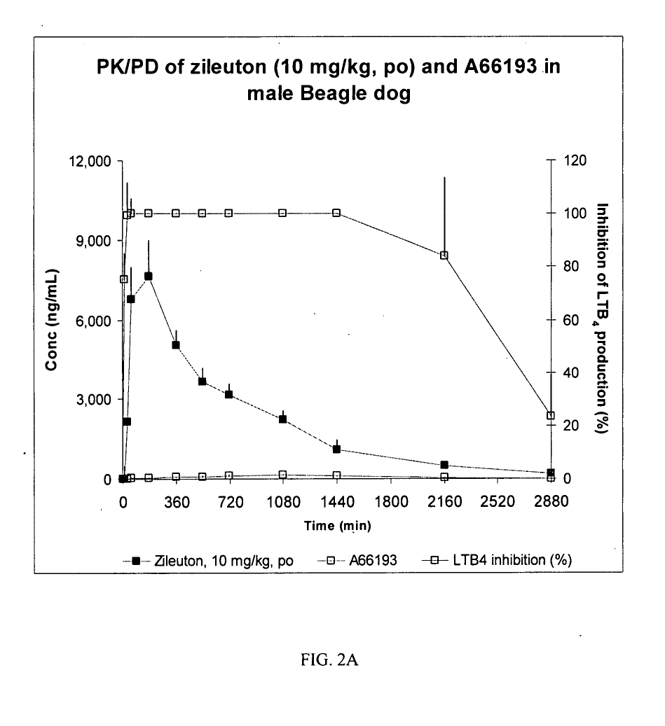

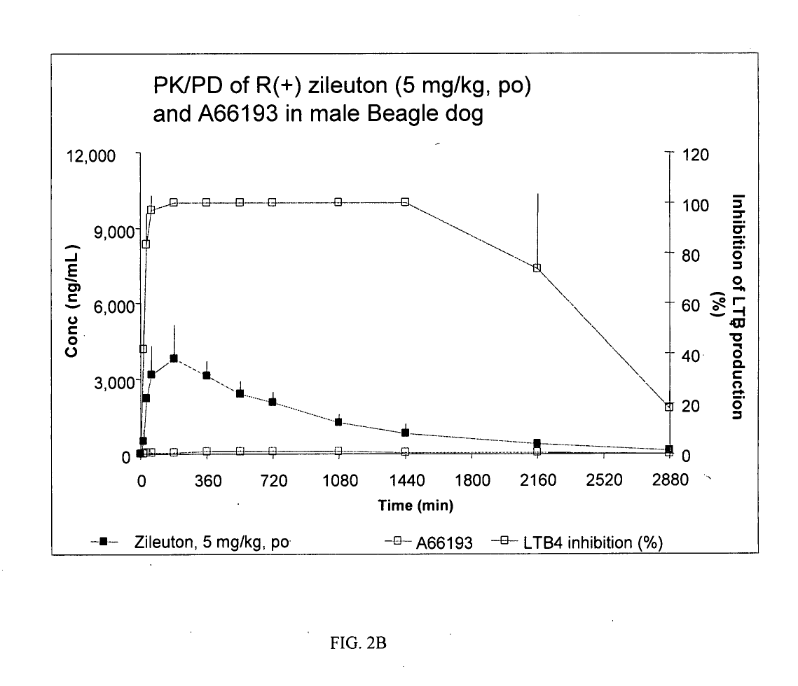

### Mean Plasma Concentration of Zileuton and Inhibition of LTB₄ Production in Whole Blood after Administration of an Oral Dose of Racemic Zileuton or (R)-Zileuton to Dogs (Methods for FIGS. 2A and 2B)

To determine the pharmacodynamics of racemic and (R)-zileuton, ex vivo, in fresh canine whole blood, a 0.5 ml aliquot was removed from each pharmacokinetic sample of the study. Each 0.5 ml aliquot of blood was placed into an individually-labeled, 1.5 ml polypropylene tube. Five (5) μl of a 5 mM calcium ionophore A23187 solution (in DMSO) was added to each sample and mixed for 10 seconds on a vortex. Final concentration of A23187 in whole blood was 50 μM (final DMSO concentration was 1%). After mixing, each sample was placed into a 37° C. circulating water bath and allowed to incubate for 30 minutes. After incubation, each sample was removed from the water bath and allowed to incubate for 30 minutes. After incubation, each sample was removed from the water bath and immediately placed onto wet ice for 2 minutes. The samples were removed from the ice after 2 minutes and immediately centrifuged in an Eppendorf Microfuge (VWR Scientific, USA) at 14,000 rpm for 2 minutes at ambient room temperature. After centrifugation, the supernatant (plasma) was collected, diluted into EIA buffer (1M phosphate buffer, pH 7.4) then stored at −80° C. until assay. The level of LTB₄ was analyzed by enzyme immunoassay (Cayman Chemical Co., Ann Arbor, Mich.).

As shown in FIG. 2A, 10 mg/kg racemic zileuton inhibited LTB₄ production by 100% at the time points measured between 30 minutes and 24 hours after administration. At 36 hours after administration, racemic zileuton inhibited LTB₄ production by about 84%. As shown in FIG. 2B, 5 mg/kg (R)-zileuton inhibited LTB₄ production by >91% at the time points measured between 60 minutes and 24 hours after administration. At 36 hours after administration, (R)-zileuton inhibited LTB₄ production by about 74%. These results demonstrate that a major component of the LTB₄ inhibition provided by racemic zileuton can be provided by one-half the dose of (R)-zileuton alone.

### Example 3

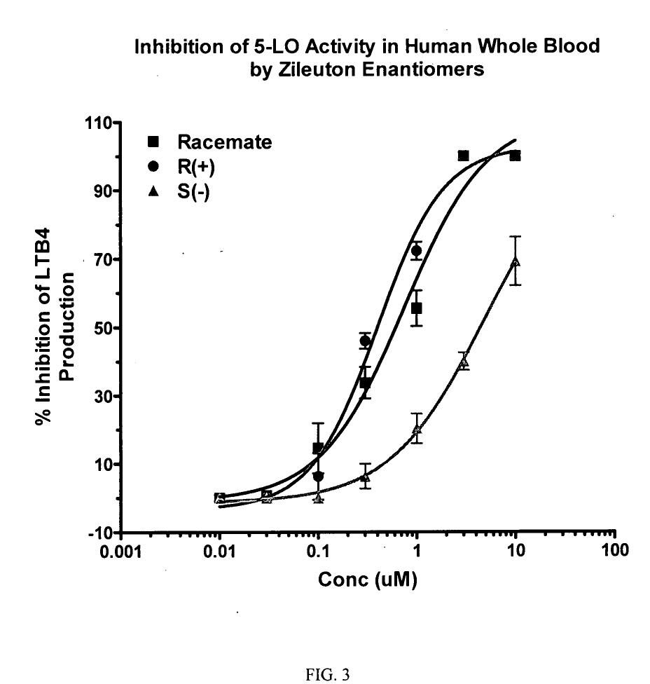

### (R)-Zileuton has Twelve-Fold Greater Potency than (S)-Zileuton in Inhibiting LTB₄ Production in Human Whole Blood Assay (Methods for FIG. 3)

The potency of racemic and (R)-zileuton was assessed in fresh human whole blood essentially by the methods of Carter et al., J. Pharm. Exp. Ther. 1991: 256:929. Briefly, 0.5.ml of heparinized (20 USP U/ml) human blood from each donor was incubated with vehicle (DMSO), or various concentrations of racemic zileuton, (R)-zileuton or (S)-zileuton (prepared in DMSO) for fifteen minutes at 37° C. Eicosanoid biosynthesis was initiated by the addition of calcium ionophore A23187 in DMSO to a final concentration of 50 μM (final DMSO concentration of 1%). Samples were incubated for an additional 30 minutes at 37° C. in the presence of A23187. The reaction was terminated by cooling in an ice bath for 2 minutes followed by centrifugation. The plasma was then salvaged to an EIA buffer (1M phosphate buffer, pH 7.4) then stored at −80° C. until assay. The level of LTB₄ was analyzed by enzyme immunoassay.

As shown in FIG. 3, in the human whole blood assay of LTB₄ inhibition, (R)-zileuton showed 12-fold greater potency in inhibiting LTB₄ production than racemic zileuton or (S)-zileuton; racemic zileuton had an IC₅₀ of 745 nM, (R)-zileuton had an IC₅₀ of 399 nM and (S)-zileuton had an IC₅₀ of 4.77 μM. The S(−) zileuton IC₅₀/R(+) zileuton IC₅₀ ratio was 11.9.

### Example 4

### Four Times Daily Treatment With Racemic Zileuton is Effective in Preventing Inflammatory Cell Infiltration in Murine Model of Allergic Lung Inflammation

Male, BALB/c mice were sensitized intraperitoneally (i.p.) with ovalbumin (10 μg) on days 1 and 14. To provoke an allergic lung inflammatory response, the mice received three intransal (i.n.) challenges with ovalbumin (100 μg) (or saline for sham) on days 29, 30 and 31. After sensitization period, animals were treated orally 30 minutes prior to ovalbumin challenge on days 29, 30 and 31 according to the following regimes: racemic Zileuton (10 mg/kg, four times daily (qid)), Montelukast (0.17 mg/kg, once daily (qd)), Dexamethasone (4 mg/kg, once daily (qd)). Twenty-four hours following the final challenge (n=16 per group, pool of two separate studies), animals were sacrificed and bronchoalveolar lavage (BAL) was performed. Infiltration of airways by inflammatory cells was quantified by cytospin, microscopy and/or flow cytometry. The dose levels and frequencies of zileuton and montelukast were chosen to approximate the human clinical use. Zileuton, Montelukast and Dexamethasone were compared to their respective “qid” and “qd” controls.

The data showing percent inhibition of cellular infiltration of airways in mice that received each type of medical intervention is presented in Table 1 below:

TABLE 1

| | Zileuton, | Montelukast, | Dexamethasone, |
|---|---|---|---|
| % Inhibition | 10 mg/kg, qid | 0.17 mg/kg, qd | 4 mg/kg, qd |
| Neutrophils | 87.9 ± 2.3 | 23.3 ± 6.6 | 90.5 ± 1.1 |
| Eosinophils | 99.2 ± 0.02 | 99.2 ± 0.02 | 99.2 ± 0.02 |
| Total cells | 62.2 ± 1.7 | 53.3 ± 6.5 | 62.5 ± 1.5 |

The data presented in Table 1 demonstrates that inhibition of airway infiltration by neutrophils by Zileuton is comparable to that by Dexamethasone and is nearly four-fold higher than that by Montelukast. Similarly, reduction by Zileuton of the total cell count was nearly the same as by Dexamethasone and almost 10% higher than that by Montelukast.

### Example 5

### R(+) Zileuton is More Active than Racemic Zileuton in Mouse Model of Asthma

Male BALB/c mice were obtained from Taconic Labs (Taconic, N.Y.) and maintained on an OVA-free (ovalbumin-free) diet. After equilibration of at least 5 days, animals were sensitized to OVA and then challenged by the following procedure. On days 1 and 14, sensitization was performed by intraperitoneal (ip) injections of 10 μg OVA emulsified in 2 mg Al(OH)₃ (aluminum hydroxide (Aldrox)) in a volume of 10 μL. On days 29, 30 and 31, allergen challenge was performed via intranasal instillation of 20 μL (10 μL/nare) of OVA (1 mg/mL) under light isoflurane anesthesia.

The background control group received no sensitization and no challenge. The sham control group underwent the OVA sensitization procedure, but was challenged with PBS. The vehicle control group was used to determine the maximal response level achievable.

Animals were given Zileuton (at 2 or 4 mg/kg, by mouth (po), once daily (qd)) or (orally) 30 minutes prior to each ovalbumin challenge. Eight hours after the final ovalbumin challenge, animals were sacrificed and bronchoalveolar lavage (BAL) was performed. A tracheal tube was inserted into the trachea and the lungs were lavaged 2 times with pyrogen-free, sterile, PBS containing 0.01% EDTA and 0.05% BSA (Bovine serum albumin). Lavage fluid, maintained at room temperature, was slowly injected into the lungs; lungs were manually massaged to distribute fluid for 30 seconds, bronchoalveolar lavage fluid (BALF) was slowly withdrawn and salvaged to individually-labeled, sterile, polypropylene tubes. The procedure was repeated 2 times/animal and the lavage fluids were pooled. The magnitude of the infiltration of inflammatory cells to the airways was determined by flow cytometry.

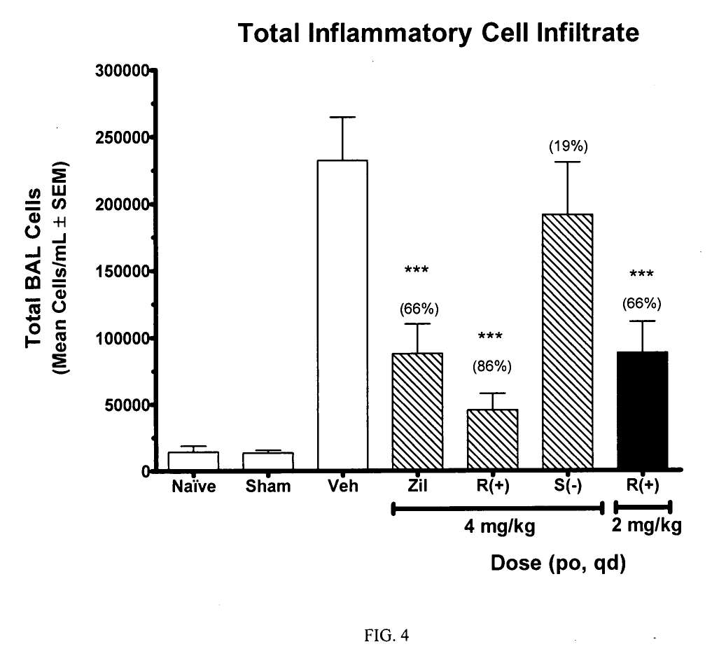

The reaction to an OVA challenge in animals previously sensitized to OVA causes a marked immunologic response encompassing a cellular influx at the site of challenge as can be seen in the vehicle control group (Veh) in FIG. 4 (The percent inhibition values of each experimental group versus the Vehicle Control group (Veh) are shown in parentheses. Statistical evaluation of the experimental groups compared with the Vehicle Control was by ANOVA with Dunnett's multiple comparison post-test (***p<0.001).) Treatment with 4 mg/kg, daily (qd) of racemic zileuton, or the R(+) enantiomer, provided a significant inhibition of the increase in the total cellularity of the BAL fluid (66% and 85% inhibition, respectively). In contrast, the S(−) enantiomer was not significantly effective in blocking the inflammatory cell infiltrate (FIG. 4). The R(+) enantiomer was also highly effective at half the dose (2 mg/kg), providing a 66% inhibition of the total cell infiltration (FIG. 4).

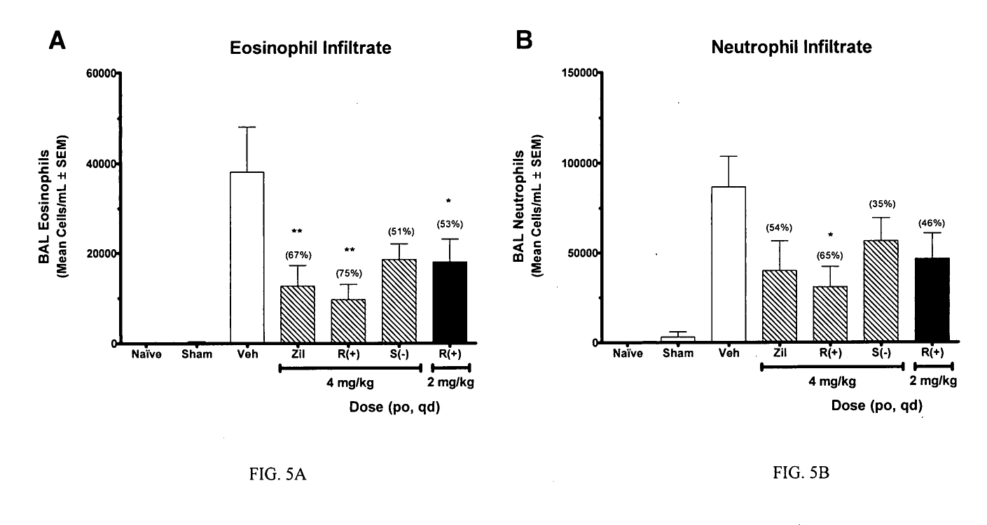

The effect of the compound treatment on the specific infiltration of eosinophils or neutrophils was evaluated by cytospin/cell counting of samples of BAL fluid. All three compounds demonstrated some inhibition of eosinophils at the 4 mg/kg dose (FIG. 5A), but only the racemate and the R(+) enantiomer provided significant inhibition (67% and 75% inhibition, respectively, versus 51% inhibition by S(−)). All three compounds showed some trend of inhibiting the neutrophil infiltration, but only the 65% inhibition seen with the R(+) at 4 mg/kg was statistically significant (FIG. 5B). Although both the racemate and S(−) provided some inhibition of the neutrophil influx (54% and 35% inhibition, respectively), these effects did not achieve statistical significance. (In both FIG. 5A and FIG. 5B, statistical evaluation of the experimental groups compared with the Vehicle Control was by ANOVA with Dunnett's multiple comparison post-test (*p<0.05; ** p<0.01).)

In summation, R(+) zileuton exhibits greater efficacy when compared to either S(−) zileuton or racemic Zileuton. Thus, administration of R(+) zileuton provides clear advantages over racemic Zileuton or S(−) zileuton as measured by in vivo efficacy in mouse model of allergen-induced airways inflammation (asthma).

While this invention has been particularly shown and described with references to example embodiments thereof, it will be understood by those skilled in the art that various changes in form and details may be made therein without departing from the scope of the invention encompassed by the appended claims.

1. A method of treating a condition selected from the group consisting of asthma, rheumatoid arthritis, gout, psoriasis, allergy, rhinitis, adult respiratory distress syndrome, chronic obstructive pulmonary disease, acne, atopic dermatitis, conjunctivitis, ischemia/reperfusion injury, atherosclerosis, aortic aneurysm, nasal polyposis, inflammatory bowel disease, irritable bowel syndrome, cancer, tumor, respiratory syncytial virus, Sjogren-Larsson syndrome, sickle cell disease, sepsis, endotoxin shock, myocardial infarction and stroke in a patient suffering therefrom comprising administering to said patient a composition comprising: i) zileuton substantially free of (S)-zileuton; and ii) a pharmaceutically acceptable excipient, wherein said zileuton is administered at a daily dose from about 450 milligrams to about 1200 milligrams per day.

2. The method of claim 1, wherein the condition is selected from the group consisting of asthma, rheumatoid arthritis, gout, psoriasis, allergy, rhinitis, adult respiratory distress syndrome, chronic obstructive pulmonary disease, acne, atopic dermatitis, ischemia/reperfusion injury, atherosclerosis, aortic aneurysm, nasal polyposis, inflammatory bowel disease, irritable bowel syndrome, cancer, tumor, respiratory syncytial virus, Sjogren-Larsson syndrome, sickle cell disease, sepsis, endotoxin shock, myocardial infarction and stroke.

3. The method of claims 1, wherein said zileuton is administered at a daily dose from about 500 milligrams to about 1000 milligrams per day.

4. The method of claim 3 wherein said zileuton is administered at a daily dose from about 600 milligrams to about 900 milligrams per day.

5. The method of claim 1, wherein said zileuton is administered at a daily dose of about 600 milligrams per day.

6. The method of claim 1, wherein said zileuton is administered at a daily dose of about 900 milligrams per day.

7. The method of claim 1 wherein said zileuton is administered at a daily dose of about 1000 milligrams per day.

8. The method of claim 1, wherein said zileuton is administered at a daily dose of about 1200 milligrams per day.

9. The method of claim 1, wherein said dose is administered orally.

10. The method of claim 1, wherein said daily dose is administered as a single daily dose.

11. The method of claim 1, wherein said daily dose is administered as two doses.

12. The method of claim 3, wherein said daily dose is administered as a single daily dose.

13. The method of claim 4, wherein said daily dose is administered as a single daily dose.

14. The method of claim 3, wherein said daily dose is administered as two doses.

15. The method of claim 4, wherein said daily dose is administered as two doses.

16. The method of claim 1, wherein said composition comprises zileuton that is at least 90% by weight (R)-zileuton and 10% by weight or less of (S)-zileuton, wherein said percent is based on the total weight of zileuton in the composition.

17-18. (canceled)

19. The method of claim 1, wherein said composition is administered as a tablet.

20. The method of claim 1, wherein said composition is administered in a controlled-release formulation.

21. The method of claim 1, wherein said condition is asthma.

22. The method of claim 21, wherein said condition is selected from the group consisting of moderate persistent asthma and severe persistent asthma.

23-38. (canceled)
